(19) United States
(12) Patent Application Publication
(10) Pub. No.: US 2025/0339352 A1
(43) Pub. Date: Nov. 6, 2025

(54) MICROEMULSION COMPOSITIONS AND RELATED METHODS

(71) Applicant: PLZ Corp., Downers Grove, IL (US)

(72) Inventor: George E. SNOW, East Taunton, MA (US)

(21) Appl. No.: 18/654,153

(22) Filed: May 3, 2024

(51) Int. Cl. A61K 8/06; A61K 8/34; A61Q 13/00

(52) U.S. Cl. CPC A61K 8/068; A61K 8/34; A61Q 13/00

(57) ABSTRACT

The present disclosure generally relates to microemulsion compositions that include an active ingredient and exhibit high clarity. The microemulsion compositions may be substantially bio-based, partially bio-based or non-bio-based and exhibit a lightness value and transmittance equal to or higher than commercially available non-bio-based microemulsion compositions. The microemulsion compositions provided herein are efficient to manufacture and maintain high clarity when subjected to high temperatures for long periods of time, freeze-thaw cycles, as well as intermediate temperatures. The microemulsion compositions provided herein produce near-universal stability with a variety of active ingredients such as oils or fragrances, thus reducing inventory costs for storage of emulsion surfactants.

### BACKGROUND OF THE DISCLOSURE

Oil-in-water emulsions are well known in many industries. In certain industries, visually appealing “clear” microemulsions are advantageous, and a litany of surfactants have been used to attempt to achieve such clear microemulsions. These microemulsions require stability under a wide range of storage conditions to be useful, as materials with a short shelf life often have to be disposed of at a great cost.

Many surfactants are composed of polymers produced from petroleum sources, and are, thus, non-renewable. Some non-renewable surfactants may be extremely expensive and subject to oil price fluctuations. Finally, some non-renewable surfactants show some weakness in freeze-thaw cycles and fail to maintain high clarity with certain compositions that are sought to be clear micro-emulsions, such as in fragrance compositions.

### SUMMARY OF THE DISCLOSURE

The present disclosure generally relates to microemulsion compositions that include an active ingredient while exhibiting high clarity. Such high clarity is sought after when microemulsion compositions are utilized in the fragrance space. The microemulsion compositions as provided herein are efficient to manufacture and maintain high clarity when subjected to high temperatures for long periods of time, freeze-thaw cycles, as well intermediate temperatures. The microemulsion compositions provided herein produce near-universal stability with a variety of active ingredients such as oils or fragrances, thus reducing inventory costs for storage of emulsion surfactants and providing an environmentally friendly composition. The microemulsion compositions as provided herein may not require non-renewable or non-bio-based surfactants. The microemulsion compositions as provided herein may utilize bio-based surfactants that are economically feasible and not subject to oil price fluctuations.

According to one aspect, a microemulsion composition is provided that includes at least one bio-based branched ethoxylated alcohol or non-bio-based branched ethoxylated alcohol; and optionally, one castor oil ethoxylate or a derivative thereof; at least one active ingredient; and water. According to one embodiment, the microemulsion composition exhibits a transmittance substantially equal to or greater than a microemulsion composition that includes or consists essentially of the same amount by weight ratio of Solubilisant LRI™ to fragrance oil. According to one embodiment, the at least one non-bio-based hydrogenated castor oil ethoxylate or a derivative thereof is present at a level of at least about 0.1% w/w based on the total weight of the microemulsion composition. According to one embodiment, the at least one non-bio-based hydrogenated castor oil ethoxylate or a derivative thereof includes 40 or more repeating units of ethylene glycol. According to one embodiment, the at least one active ingredient is a fragrance selected from the group consisting of White Cashmere, Sea Minerals, Bamboo Blossom, Champagne Toast, Warm and Cozy, or other fragrance/oil. According to one embodiment, the microemulsion composition has an average emulsion droplet size of 80 nm or less. According to one embodiment, the microemulsion composition has an average emulsion droplet size of 60 nm or less. According to one embodiment, the microemulsion composition further comprises at least one antifoam solution, at least one preservative, ethanol, isopropanol, a glycol of below 300 molecular weight, or a combination thereof.

According to one aspect, a microemulsion composition is provided that includes at least one bio-based branched ethoxylated alcohol; and optionally at least one bio-based alkyl glucoside alone or in combination with at least one linear ethoxylated alcohol; at least one active ingredient; and water. According to one embodiment, the microemulsion composition exhibits a transmittance substantially equal to or greater than a microemulsion composition that includes or consists essentially of the same amount by weight ratio of Solubilisant LRI™ to fragrance oil. According to one embodiment, the microemulsion composition exhibits a transmittance of at least 90%, 91%, 92%, 93%, 94%, 95%, 96%, or 97%. According to one embodiment, the at least one bio-based ethoxylated alcohol and at least one bio-based alkyl glucoside each exhibit a hydrophilic-lipophilic balance (HLB) of at least about 6. According to one embodiment, the microemulsion includes a bio-based branched ethoxylated isocetyl alcohol; a bio-based linear ethoxylated alcohol; and a bio-based sorbitan oleate decyl glucoside. According to one embodiment, the microemulsion composition has a ratio of bio-based branched ethoxylated isocetyl alcohol to bio-based linear ethoxylated alcohol to bio-based sorbitan oleate decyl glucoside of about 65:15:25. According to one embodiment, the microemulsion further includes at least one antifoam solution, at least one preservative, ethanol, or a combination thereof. According to one embodiment, the at least one active ingredient is a fragrance selected from the group consisting of White Cashmere, Sea Minerals, Bamboo Blossom, Champagne Toast, Warm and Cozy, or other fragrance/oil. According to one embodiment, the microemulsion composition has an average emulsion droplet size of 80 nm or less. According to one embodiment, the microemulsion composition includes a total bio-based content of at least about 97% w/w based on the total weight of the microemulsion composition. According to one embodiment, the at least one bio-based branched ethoxylated alcohol is present in an amount of at least about 1.0% w/w based on a total weight percent of the at least one bio-based branched ethoxylated alcohol and at least one bio-based alkyl glucoside alone or in combination at least one linear ethoxylated alcohol.

According to one aspect, a microemulsion composition is provided that includes at least one branched ethoxylated alcohol; at least one hydrogenated castor oil or derivative thereof including 40 or more repeating units of ethylene glycol; at least one active ingredient; and water. According to one embodiment, the microemulsion composition exhibits a transmittance substantially equal to or greater than a microemulsion composition that includes or consists essentially of the same amount by weight ratio of Solubilisant LRI™ to fragrance oil According to one embodiment, the at least one hydrogenated castor oil or a derivative thereof is a polyethylene glycol derivative of hydrogenated castor oil. According to one embodiment, the at least one active ingredient is a fragrance selected from the group consisting of White Cashmere, Sea Minerals, Bamboo Blossom, Champagne Toast, Warm and Cozy, or other fragrance/oil. According to one embodiment, the microemulsion composition has an average emulsion droplet size of 80 nm or less. According to one embodiment, the microemulsion composition has an average emulsion droplet size of 60 nm or less.

### DETAILED DESCRIPTION

The present disclosure will now be described more fully hereinafter with reference to exemplary embodiments thereof. These exemplary embodiments are described so that this disclosure will be thorough and complete, and will fully convey the scope of the disclosure to those skilled in the art. Indeed, the present disclosure may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

### Definitions

As used herein, the term “bio-based” refers to the state of being manufactured from or derived (wholly or in significant part) from a renewable source. The renewable source may typically be a carbon source that was previously living (e.g., plant source). Bio-based microemulsion components described herein are not manufactured from, derived from or in any way include petroleum-based components.

As used herein, the term “non-bio-based” refers to the state of being manufactured from or derived (whole or in significant part) from a non-renewable source (e.g. petroleum based).

As used herein, the term “partially bio-based” refers to the state of being manufactured from or derived (whole or in significant part) from a renewable source as well as a non-renewable source (e.g. petroleum based).

As used herein, the term “L value” or “lightness value” refers to the clarity of a sample on a scale of 0-100 as determined by a colorimeter utilizing standard testing methods. A value of “0” refers to a lightness value reading from a completely dark sample. A value of 100 refers to a lightness value reading from a completely diffuse sample such that light entering a sample is equal to the value of light exiting a sample.

As used herein, the term “transmittance (T)” refers to a fraction of incident light which is transmitted or successfully passes through a liquid sample and mathematically defined as T=I/Io, where I=transmitted light (“output”) and Io =incident light (“input”). Transmittance may be measured using a light-sensitive device such as a colorimeter.

As used herein, the “percentage (%) transmittance” is merely (I/Io)×100. For example, if T=0.25, then % T=25%. A % T of 25% would indicate that 25% of the light passed through the sample and emerged on the other side.

As used herein, the term “microemulsion” refers to a stable, substantially clear mixture of at least one surfactant and an oil-based active ingredient. The microemulsions as provided herein are formed under low shear (i.e., not formed under high shear).

As used herein, the term “surfactant” refers to a surface active amphiphilic group of atoms.

As used herein, the product Solubilisant LRI™ refers to a surfactant blend that includes, in part, or consists essentially of PPG-26-Buteth-26 (2-(1-butoxypropan-2-yloxy)ethanol) and PEG-40 hydrogenated castor oil.

As used herein, “PPG-26-Buteth-26” refers to a polyoxypropylene, polyoxyethylene ether of butyl alcohol—particularly, 2-(1-butoxypropan-2-yloxy) ethanol.

As used herein, “PEG-40 hydrogenated castor oil” refers to a combination of synthetic polyethylene glycol with bio-based castor oil. PEG-40 hydrogenated castor oil (CAS 61788-85-0) is produced via ethoxylation via addition of 40 ethylene oxide units to hydrogenated castor oil.

### Microemulsion Composition Overview

The present disclosure generally relates to microemulsion compositions that include an active ingredient and exhibit high clarity. The microemulsion compositions may be substantially bio-based, partially bio-based or non-bio-based and exhibit a lightness value and transmittance equal to or higher than commercially available non-bio-based microemulsion compositions. The microemulsion compositions provided herein are efficient to manufacture and maintain high clarity when subjected to high temperatures for long periods of time, freeze-thaw cycles, as well intermediate temperatures. The microemulsion compositions provided herein produce near “universal” stability with a variety of active ingredients such as oils or fragrances, thus reducing inventory costs for storage of emulsion surfactants.

### Ethoxylated Alcohol

The microemulsion compositions provided herein may include at least one branched ethoxylated alcohol. The at least one branched ethoxylated alcohol included in the microemulsion compositions provided herein may be bio-based or non-bio-based. The ethoxylated alcohols described herein may optionally contain linear ethoxylated alcohols. The ethoxylated alcohols described herein may be surfactants.

According to one embodiment, the at least one ethoxylated alcohol exhibits a hydrophilic-lipophilic balance (HLB) that is suitable for use in combination with any active ingredient as described herein. According to one embodiment, the at least one ethoxylated alcohol exhibits a hydrophilic-lipophilic balance (HLB) of at least about 6. According to one embodiment, the at least one ethoxylated alcohol exhibits a hydrophilic-lipophilic balance (HLB) of greater than 6. According to one embodiment, the at least one ethoxylated alcohol exhibits a hydrophilic-lipophilic balance (HLB) of at least about 7. According to one embodiment, the at least one ethoxylated alcohol exhibits a hydrophilic-lipophilic balance (HLB) of at least about 8. According to one embodiment, the at least one ethoxylated alcohol exhibits a hydrophilic-lipophilic balance (HLB) of at least about 9. According to one embodiment, the at least one ethoxylated alcohol exhibits a hydrophilic-lipophilic balance (HLB) of at least about 10.

The at least one ethoxylated alcohol may be a non-ionic surfactant that functions as a solubilizer and emulsifier. The at least one ethoxylated alcohol may be bio-based and include polyoxyethylene vegetable-based fatty ethers derived from iso-lauryl, iso-cetyl, iso-stearyl and iso-oleyl alcohols. According to a particular embodiment, the at least one bio-based, branched ethoxylated alcohol is an ethoxylated isocetyl alcohol such as Brij™ IC20-70 available from Croda. A suitable example of a non-bio-based, branched ethoxylated alcohol includes Tergitol™ 15-S-12 available from Dow.

The microemulsion compositions provided herein may include at least one linear ethoxylated alcohol. A suitable example of a bio-based linear ethoxylated alcohol includes Linsurf™ LA-9 85 NAT available from Barentz. The microemulsion compositions provided herein may include at least one bio-based isotridecyl ethoxylate. The microemulsion compositions provided herein may include at least one ethoxylated alcohol that is a primary alcohol with beta branching (i.e., Guerbet alcohol).

According to one embodiment, the at least one ethoxylated alcohol includes at least about 5 ethoxylate units. According to one embodiment, the at least one ethoxylated alcohol includes at least about 9 ethoxylate units. According to one embodiment, the at least one ethoxylated alcohol includes at least about 15 ethoxylate units. According to one embodiment, the at least one ethoxylated alcohol includes at least about 20 ethoxylate units. According to one embodiment, the at least one ethoxylated alcohol includes at least about 25 ethoxylate units. According to one embodiment, the at least one ethoxylated alcohol includes at least about 30 ethoxylate units. According to one embodiment, the at least one ethoxylated alcohol includes at least about 35 ethoxylate units. According to one embodiment, the at least one ethoxylated alcohol includes 40 or more ethoxylate units.

According to one embodiment, the microemulsion compositions provided herein include at least one branched ethoxylated alcohol in an amount of about 0.1% w/w based on the weight of the microemulsion composition. According to one embodiment, the microemulsion compositions provided herein include at least one branched ethoxylated alcohol in an amount of about 1.0% w/w based on the weight of the microemulsion composition. According to one embodiment, the microemulsion compositions provided herein include at least one branched ethoxylated alcohol in an amount of about 1.5% w/w based on the weight of the microemulsion composition. According to one embodiment, the microemulsion compositions provided herein include at least one branched ethoxylated alcohol in an amount of about 2.0% w/w based on the weight of the microemulsion composition. According to one embodiment, the microemulsion compositions provided herein include at least one branched ethoxylated alcohol in an amount of about 3.0% w/w based on the weight of the microemulsion composition. According to one embodiment, the microemulsion compositions provided herein include at least one branched ethoxylated alcohol in an amount of about 4.0% w/w based on the weight of the microemulsion composition. According to one embodiment, the microemulsion compositions provided herein include at least one branched ethoxylated alcohol in an amount of about 5.0% w/w based on the weight of the microemulsion composition. According to one embodiment, the microemulsion compositions provided herein include at least one branched ethoxylated alcohol in an amount of about 6.0% w/w based on the weight of the microemulsion composition. According to one embodiment, the microemulsion compositions provided herein include at least one branched ethoxylated alcohol in an amount of about 7.0% w/w based on the weight of the microemulsion composition. According to one embodiment, the microemulsion compositions provided herein include at least one branched ethoxylated alcohol in an amount of about 8.0% w/w based on the weight of the microemulsion composition.

### Alkyl Glucoside

The microemulsion compositions provided herein may include at least one alkyl glucoside. The alkyl glucosides as provided herein are bio-based.

According to one embodiment, the at least one alkyl glucoside exhibits a hydrophilic-lipophilic balance (HLB) that is suitable use in combination with any active ingredient as described herein. According to one embodiment, the at least one alkyl glucoside exhibits a hydrophilic-lipophilic balance (HLB) of at least about 6. According to one embodiment, the at least one alkyl glucoside exhibits a hydrophilic-lipophilic balance (HLB) of greater than 6. According to one embodiment, the at least one alkyl glucoside exhibits a hydrophilic-lipophilic balance (HLB) of at least about 7. According to one embodiment, the at least one alkyl glucoside exhibits a hydrophilic-lipophilic balance (HLB) of at least about 8. According to one embodiment, the at least one alkyl glucoside exhibits a hydrophilic-lipophilic balance (HLB) of at least about 9. According to one embodiment, the at least one alkyl glucoside exhibits a hydrophilic-lipophilic balance (HLB) of at least about 10.

According to one embodiment, the at least one alkyl glucoside is a nonionic fragrance solubilizer. According to one embodiment, the at least one alkyl glucoside is a bio-based sorbitan oleate decyl glucoside. A suitable example of a bio-based sorbitan oleate decyl glucoside is Poly Suga®Mulse D9 available from Colonial Chemical.

According to one embodiment, the at least one alkyl glucoside is ethylene-oxide (EO) free (i.e., does not contain EO units). According to one embodiment, the at least one alkyl glucoside is bio-based and polyethylene glycol free (i.e., does not contain polyethylene glycol). According to one embodiment, the at least one alkyl glucoside is bio-based and 1,4-dioxane free (i.e., does not contain 1,4-dioxane). According to one embodiment, the at least one alkyl glucoside is bio-based and hydrophilic. According to one embodiment, the at least one alkyl glucoside is bio-based and freely soluble or dispersible in water.

According to one embodiment, the microemulsion compositions provided herein include at least one bio-based alkyl glucoside in an amount of from about 0.1% w/w to about 98% w/w of total surfactant content. According to one embodiment, the microemulsion compositions provided herein include at least one bio-based alkyl glucoside in an amount of from about 0.1% w/w to about 60% w/w of total surfactant content. According to one embodiment, the microemulsion compositions provided herein include at least one bio-based alkyl glucoside in an amount of less than 60% w/w of total surfactant content.

### Castor Oil or Derivative

The microemulsion compositions provided herein may include at least one ethoxylated castor oil derivative. Any of the at least one castor oil derivatives thereof as provided herein may be bio-based. According to one embodiment, the at least one castor oil is a partially bio-based hydrogenated castor oil. A suitable example of a bio-based hydrogenated castor oil is Ethoxcare™ HCO-60 available from Ethox.

According to one embodiment, the microemulsion compositions provided herein may include at least one castor oil derivative that is a glycol of a hydrogenated castor oil with an average of 40 or greater than 40 repeating units of ethylene glycol. According to one embodiment, the at least one castor oil derivative is a glycol of a hydrogenated castor oil with an average of about 50 repeating units of ethylene glycol. According to one embodiment, the at least one castor oil derivative is a glycol of a hydrogenated castor oil with an average of about 60 repeating units of ethylene glycol.

According to one embodiment, the microemulsion compositions provided herein may include at least one glycol that is a non-volatile organic compound (VOC) glycol of molecular weight less than 1000 Daltons. According to one embodiment, the at least one glycol that is a non-VOC glycol, is of a molecular weight less than 900 Daltons. According to one embodiment, the at least one glycol that is a non-VOC glycol, is of a molecular weight less than 800 Daltons. According to one embodiment, the at least one glycol is a non-VOC glycol is of a molecular weight less than 700 Daltons. According to one embodiment, the at least one glycol that is a non-VOC glycol is of a molecular weight less than 600 Daltons. According to one embodiment, the at least one glycol is a non-VOC glycol is of a molecular weight less than 500 Daltons. Exemplary embodiments of the at least one glycol that is a non-volatile organic compound (VOC) glycol of molecular weight less than 1000 Daltons include, but are not limited to, propylene glycol, di-propylene glycol, diethylene glycol, glycerin, 1,2 butylene glycol, 1,3 butylene glycol, 1,4 butylene glycol, or ethyl-hexyl glycerin.

According to one embodiment, the microemulsion compositions provided herein may include at least one castor oil or a derivative thereof that is a pegylated hydrogenated castor oil. According to one embodiment, the pegylated hydrogenated castor oil exhibits an average polyethylene glycol (PEG) content of 40 or more units. According to one embodiment, the microemulsion compositions provided herein may include at least one castor oil or a derivative thereof that is a non-hydrogenated castor oil. According to one embodiment, the microemulsion compositions provided herein may include at least one castor oil or a derivative thereof that is an ethoxylated hydrophobe, or other branched hydrophobes that are comb structured.

According to one embodiment, the microemulsion compositions provided herein include at least one castor oil or a derivative thereof in an amount of about 0.1% w/w based on the total weight of the microemulsion composition. According to one embodiment, the microemulsion compositions provided herein include at least one castor oil or a derivative thereof in an amount of about 0.5% w/w. According to one embodiment, the microemulsion compositions provided herein include at least one castor oil or a derivative thereof in an amount of about 1.0% w/w of total formula. According to one embodiment, the microemulsion compositions provided herein include at least one castor oil or a derivative thereof in an amount of about 1.5% w/w of total formula. According to one embodiment, the microemulsion compositions provided herein include at least one castor oil or a derivative thereof in an amount of about 2.0% w/w of total formula. According to one embodiment, the microemulsion compositions provided herein include at least one castor oil or a derivative thereof in an amount of about 2.5% w/w of total formula. According to one embodiment, the microemulsion compositions provided herein include at least one castor oil or a derivative thereof in an amount of about 3.0% w/w of total formula. According to one embodiment, the microemulsion compositions provided herein include at least one castor oil or a derivative thereof in an amount of about 3.5% w/w of total formula. According to one embodiment, the microemulsion compositions provided herein include at least one castor oil or a derivative thereof in an amount of about 4.0% w/w of total formula. According to one embodiment, the microemulsion compositions provided herein include at least one castor oil or a derivative thereof in an amount of about 4.5% w/w of total formula. According to one embodiment, the microemulsion compositions provided herein include at least one castor oil or a derivative thereof in an amount of about 5.0% w/w of total formula. According to one embodiment, the microemulsion compositions provided herein include at least one castor oil or a derivative thereof in an amount of about 5.5% w/w of total formula. According to one embodiment, the microemulsion compositions provided herein include at least one castor oil or a derivative thereof in an amount of about 6.0% w/w of total formula.

### Active Ingredients and Methods of Use

The microemulsion compositions provided herein include at least one active ingredient. According to one embodiment, the at least one ingredient may be an oil. According to one embodiment, the at least one ingredient may be a fragrance. The fragrance may be formulated as an oil. According to one embodiment, the fragrance is White Cashmere, Sea Minerals, Bamboo Blossom, Champagne Toast, Warm and Cozy, or other fragrance/oil, or a combination thereof.

According to one embodiment, the microemulsion compositions provided herein include at least one active ingredient in an amount of about 0.01% w/w based on the total weight of the microemulsion composition. According to one embodiment, the microemulsion compositions provided herein include at least one active ingredient in an amount of about 0.1% w/w based on the total weight of the microemulsion composition. According to one embodiment, the microemulsion compositions provided herein include at least one active ingredient in an amount of up to 0.5% w/w. According to one embodiment, the microemulsion compositions provided herein include at least one active ingredient in an amount of up to 1.0% w/w. According to one embodiment, the microemulsion compositions provided herein include at least one active ingredient in an amount of up to 1.5% w/w. According to one embodiment, the microemulsion compositions provided herein include at least one active ingredient in an amount of up to 2.0% w/w. According to one embodiment, the microemulsion compositions provided herein include at least one active ingredient in an amount of up to 2.5% w/w. According to one embodiment, the microemulsion compositions provided herein include at least one active ingredient in an amount of up to 5.0% w/w. According to one embodiment, the microemulsion compositions provided herein include at least one active ingredient in an amount of up to 8.0% w/w. According to one embodiment, the microemulsion compositions provided herein include at least one active ingredient in an amount of up to about 15.0% w/w.

According to one embodiment, the microemulsion compositions provided herein include at least one active ingredient in an amount of from about 0.01% w/w to about 40% w/w based on the total weight of the microemulsion composition. According to one embodiment, the microemulsion compositions provided herein include at least one active ingredient in an amount of from about 0.1% w/w to about 35% w/w based on the total weight of the microemulsion composition. According to one embodiment, the microemulsion compositions provided herein include at least one active ingredient in an amount of from about 1.00% w/w to about 30% w/w based on the total weight of the microemulsion composition. According to one embodiment, the microemulsion compositions provided herein include at least one active ingredient in an amount of from about 1.00% w/w to about 27.5% w/w based on the total weight of the microemulsion composition. According to one embodiment, the microemulsion compositions provided herein include at least one active ingredient in an amount of from about 1.00% w/w to about 25% w/w based on the total weight of the microemulsion composition. According to one embodiment, the microemulsion compositions provided herein include at least one active ingredient in an amount of from about 1.00% w/w to about 20% w/w based on the total weight of the microemulsion composition.

### Other Components of the Microemulsion Compositions

The microemulsion compositions provided herein may include at least one additional component. Any or all of the additional components may be bio-based.

According to one embodiment, the microemulsion compositions provided herein include water. The water may be deionized, distilled, or otherwise treated or untreated.

According to one embodiment, the microemulsion compositions provided herein may include at least one antifoam solution. The antifoam solution may be bio-based.

According to one embodiment, the microemulsion compositions provided herein may include at least one preservative. The preservative may be bio-based.

According to one embodiment, the microemulsion compositions provided herein may include various additional surfactants, including nonionic surfactants, anionic surfactants, cationic surfactants, and amphoteric surfactants.

### Microemulsion Composition Characteristics

The various microemulsion composition components described herein may be substantially or completely bio-based in origin. The percentage of bio-based content is based on the weight of all components included in the microemulsion composition. According to one embodiment, the microemulsion compositions provided herein may include a total bio-based content of at least about 80% w/w. According to one embodiment, the microemulsion compositions provided herein may include a total bio-based content of at least about 85% w/w. According to one embodiment, the microemulsion compositions provided herein may include a total bio-based content of at least about 90% w/w. According to one embodiment, the microemulsion compositions provided herein may include a total bio-based content of at least about 95% w/w. According to one embodiment, the microemulsion compositions provided herein may include a total bio-based content of at least about 96% w/w. According to one embodiment, the microemulsion compositions provided herein may include a total bio-based content of at least about 97% w/w. According to one embodiment, the microemulsion compositions provided herein may include a total bio-based content of at least about 98% w/w. According to one embodiment, the microemulsion compositions provided herein may include a total bio-based content of at least about 99% w/w. According to one embodiment, the microemulsion compositions provided herein may include a total bio-based content of about 100% w/w.

According to a particular embodiment, the microemulsion compositions provided herein may include zero or no bio-based surfactant content. According to such an embodiment, the microemulsion compositions include a branched ethoxylated alcohol, and potentially including an ethoxylated castor oil derivative with 40 or more ethylene oxide units.

According to one embodiment, the various microemulsion composition components, with the exception of the active ingredient, may include little to no volatile organic compounds (VOC). According to one embodiment, the various microemulsion composition components, with the exception of the active ingredient, may be completely free of volatile organic compounds (VOC). According to one embodiment, the various microemulsion composition components, with the exception of the active ingredient, may include less than about 8% w/w volatile organic compounds (VOC). According to one embodiment, the various microemulsion composition components, with the exception of the active ingredient, may include less than about 4% w/w volatile organic compounds (VOC). According to one embodiment, the various microemulsion composition components, with the exception of the active ingredient, may include less than about 1% w/w volatile organic compounds (VOC).

According to one embodiment, the microemulsion compositions provided herein exhibit a transmittance percentage about equal to, or greater than, microemulsions generated by using Solubilisant LRI™.

According to one embodiment, the microemulsion compositions provided herein exhibit a transmittance percentage of at least about 90.0%. According to one embodiment, the microemulsion compositions provided herein exhibit a transmittance percentage of at least about 91.0%. According to one embodiment, the microemulsion compositions provided herein exhibit a transmittance percentage of at least about 92.0%. According to one embodiment, the microemulsion compositions provided herein exhibit a transmittance percentage of at least about 93.0%. According to one embodiment, the microemulsion compositions provided herein exhibit a transmittance percentage of at least about 94.0%. According to one embodiment, the microemulsion compositions provided herein exhibit a transmittance percentage of at least about 95.0%. According to one embodiment, the microemulsion compositions provided herein exhibit a transmittance percentage of at least about 96.0%. According to one embodiment, the microemulsion compositions provided herein exhibit a transmittance percentage of at least about 97.0%.

According to one embodiment, the microemulsion compositions provided herein exhibit a lightness value about equal to, or greater than, microemulsions generated by using Solubilisant LRI™. According to one embodiment, the microemulsion compositions provided herein exhibit a lightness value of at least about 90.0%. According to one embodiment, the microemulsion compositions provided herein exhibit a lightness value of at least about 91.0%. According to one embodiment, the microemulsion compositions provided herein exhibit a lightness value of at least about 92.0%. According to one embodiment, the microemulsion compositions provided herein exhibit a lightness value of at least about 93.0%. According to one embodiment, the microemulsion compositions provided herein exhibit a lightness value of at least about 94.0%. According to one embodiment, the microemulsion compositions provided herein exhibit a lightness value of at least about 95.0%. According to one embodiment, the microemulsion compositions provided herein exhibit a lightness value of at least about 96.0%. According to one embodiment, the microemulsion compositions provided herein exhibit a lightness value of at least about 97.0%.

According to one embodiment, the microemulsion compositions provided herein exhibit an average emulsion droplet size of 80 nm or less. According to one embodiment, the microemulsion compositions provided herein exhibit an average emulsion droplet size of 75 nm or less. According to one embodiment, the microemulsion compositions provided herein exhibit an average emulsion droplet size of 70 nm or less. According to one embodiment, the microemulsion compositions provided herein exhibit an average emulsion droplet size of 65 nm or less. According to one embodiment, the microemulsion compositions provided herein exhibit an average emulsion droplet size of 60 nm or less. According to one embodiment, the microemulsion compositions provided herein exhibit an average emulsion droplet size of 55 nm or less. According to one embodiment, the microemulsion compositions provided herein exhibit an average emulsion droplet size of 50 nm or less. According to one embodiment, the microemulsion compositions provided herein exhibit an average emulsion droplet size of 45 nm or less. According to one embodiment, the microemulsion compositions provided herein exhibit an average emulsion droplet size of 40 nm or less.

### Methods of Preparation

Methods of manufacturing or otherwise preparing a microemulsion composition are provided. According to one embodiment, the method of preparing a microemulsion composition includes the step of forming an oil phase. The step of forming an oil phase includes the initial step of combining at least one bio-based ethoxylated alcohol and at least one active ingredient to form a solution. According to one embodiment, the method includes the step of mixing the solution. The mixing may be provided by agitation such as via a magnetic stir bar or other acceptable mixing device. The step of forming an oil phase may also include the step of introducing at least one castor oil or a derivative thereof to the solution to form the oil phase. According to one embodiment, the method includes the step of mixing the oil phase. The mixing may be provided by agitation such as via a magnetic stir bar, or other suitable method.

According to one embodiment, the method of preparing a microemulsion composition includes the step of forming an aqueous phase. The step of forming an aqueous phase including the step of optionally mixing deionized water with one or more of a preservative, antifoam solution, or ethanol. According to one embodiment, the aqueous phase includes only deionized water.

According to one embodiment, the method of preparing a microemulsion composition includes the step of combining the aqueous phase and oil phase to form the microemulsion composition. The step of combining the aqueous phase and oil phase may be performed under low shear. The step of combining the aqueous phase and oil phase is not performed under high shear. The step of combining the aqueous phase and oil phase may include mixing the aqueous phase and oil phase for up to, at least 30 minutes. The step of combining the aqueous phase and oil phase may include mixing the aqueous phase and oil phase for less than 30 minutes. The step of combining the aqueous phase and oil phase may include mixing the aqueous phase and oil phase for about 5 minutes. The step of combining the aqueous phase and oil phase may include mixing the aqueous phase and oil phase for about 10 minutes. The step of combining the aqueous phase and oil phase may include mixing the aqueous phase and oil phase for about 15 minutes. The step of combining the aqueous phase and oil phase may include mixing the aqueous phase and oil phase for about 20 minutes. The step of combining the aqueous phase and oil phase may include mixing the aqueous phase and oil phase for about 25 minutes.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included within the scope of the appended claims.

Clarity measurements (L value) were taken using a Konica-Minolta™ CM-5 colorimeter. The cuvettes used were 1 cm in length/width, and held 4.5 ml of liquid. The composition of the cuvettes was acrylic. Illuminant C was the light source.

Deionized (DI) water gave lightness value (L) readings of between 96.75 and 97.07 throughout the testing, and gave an example of the ultimate potential clarity of a micro-emulsion system. Readings of above 96 are considered exceptional in terms of clarity, while readings of 95 and above are considered very good. Readings of between 94 and 95 would be considered good. Readings between 90 and 94 would generally be considered fair to sub-par. Readings below 90 would generally be considered undesirable. Visual performance is understood to be somewhat subjective and may vary between consumers, producers, and marketers/sellers.

The clarity values of the tested emulsions are listed in the following tables, each utilizing five different fragrances at 2.00% w/w were compared. The tables listed below, show the results of this clarity comparison initially, after three freeze/thaw cycles, at two (2) months after samples were held at 45° C., and at two (2) months after samples were held at 5° C. The freeze/thaw process included placing samples in a freezer at −20° C. for about 14 hours. At the end of 14 hours, samples were allowed to warm to room temperature (about 21 C) for a minimum of 8 hours. The haze values in the following tables were read after three freeze/thaw cycles. All samples were allowed to equilibrate to room temperature, about 21 C, before measurements were taken.

In the following examples, batch sizes were 200 grams.

### Inventive Example 1 (Partial Bio-based)

The microemulsion compositions of the present example included each of the components and amounts as set forth in EXAMPLE 1 (referred to as Partially Bio-based Microemulsion—Composition #1).

### Example 1.

### Partially Bio-based Microemulsion—Composition #1

| Component | Weight/Weight Percentage (%) |
|---|---|
| Brij ™ IC20-70 | 2.50 |
| PEG-60 Hydrogenated Castor | 3.25 |
| Oil (partially bio-based) | |
| Fragrance | 2.00 |
| Preservative | 0.65 |
| 1% Antifoam Solution | 0.50 |
| DI Water | 91.10 |

To prepare the Partially Bio-based Microemulsion Composition #1 of the present example, Brij™ IC20-70, a bio-based branched ethoxylated alcohol having a hydrophilic-lipophilic balance of greater than 6, was weighed into a glass beaker. Agitation with a magnetic stir bar was started at 500 rpm. A partially bio-based ethoxylated hydrogenated castor oil (Ethoxcare™ HCO-60, a PEG-60 Hydrogenated Castor Oil, or PEG-60 HCO) was warmed to 60° C. for melting and added under agitation. Once homogeneous, the fragrance was added. A preservative, antifoam, and Deionized (DI) water were then sequentially added to form the Partially Bio-based Microemulsion Composition #1. The Partially Bio-based Microemulsion Composition #1 was mixed for a minimum of 30 minutes. The Partially Bio-based Microemulsion Composition #1 was observed to have generally very good transparency, and did not likely need a full 30 minutes of agitation. Results are seen below in table 1.

TABLE 1
Partially Bio-based Microemulsion Inventive Examples #1-#5

| | | | RT | 2 Mo | 2 Mo |
|---|---|---|---|---|---|
| | | F/T | (21° C.) | 45° C. | 5° C. |
| Microemulsion Composition | Fragrance | Haze L | Haze L | Haze L | Haze L |
| Control (LRI) | White Cashmere | 88.29 | 95.80 | 91.98 | 95.93 |
| Partially Bio-based Example | White Cashmere | 95.67 | 95.93 | 96.29 | 96.22 |
| Control (LRI) | Sea Minerals | 83.28 | 95.98 | 87.33 | 96.06 |
| Partially Bio-based Example | Sea Minerals | 95.86 | 96.04 | 96.15 | 96.17 |
| Control (LRI) | Bamboo Blossom | 73.27 | 95.34 | 66.34 | 95.41 |
| Partially Bio-based Example | Bamboo Blossom | 85.01 | 96.17 | 95.51 | 96.26 |
| Control (LRI) | Champagne Toast | 95.09 | 95.81 | 90.03 | 95.67 |
| Partially Bio-based Example | Champagne Toast | 95.50 | 95.58 | 95.86 | 95.80 |
| Control (LRI) | Warm and Cozy | 82.50 | 95.97 | 92.67 | 96.19 |
| Partially Bio-based Example | Warm and Cozy | 93.53 | 96.29 | 96.48 | 96.41 |

As can be seen from Table 1, the clarity of each of the fragrance compositions utilizing the Partially Bio-based Microemulsion Composition #1 were generally better than or equal to the fragrance compositions utilizing the Control LRI surfactant.

### Inventive Example 2

The microemulsion compositions of the present example included each of the components and amounts as set forth in Example 2 (referred to as Low Cost Microemulsion—Composition #2).

### Example 2.

### Low Cost Microemulsion—Composition #2

| Component | Weight/Weight Percentage (%) |
|---|---|
| Tergitol ™ 15-S-12 | 3.25 |
| Fragrance | 2.00 |
| PEG-60 Hydrogenated | 1.75 |
| Castor Oil | |
| Preservative | 0.65 |
| 1% Antifoam Solution | 0.50 |
| DI Water | 91.85 |

The branched ethoxylated alcohol (Tergitol™ 15-S-12) was warmed to 45° C. for melting, and was weighed into a glass beaker. Agitation was started with a magnetic stir bar at 500 rpm. The PEG-60 hydrogenated castor oil (Ethoxcare™ HCO-60) was warmed to 60° C. for melting and added to the beaker under agitation. Once homogeneous, the fragrance was added. Then, a preservative, antifoam, and Deionized (DI) water were added sequentially to form the Low Cost Microemulsion Composition #2. The Low Cost Microemulsion Composition #2 was mixed for a minimum of 30 minutes at 500 rpm. The Low Cost Microemulsion Composition #2 was observed to have generally very good transparency, and did not likely need a full 30 minutes of agitation. Results are seen below in table 2.

### Table 2.

### Low Cost Microemulsion Inventive Examples #6-#10

| | | | RT | 2 Mo | 2 Mo |
|---|---|---|---|---|---|
| | | F/T | (21° C.) | 45° C. | 5° C. |
| Microemulsion Composition | Fragrance | Haze L | Haze L | Haze L | Haze L |
| Control (LRI) | White Cashmere | 88.29 | 95.80 | 91.98 | 95.93 |
| Low Cost Example | White Cashmere | 96.21 | 96.17 | 95.39 | 96.13 |
| Control (LRI) | Sea Minerals | 83.28 | 95.98 | 87.33 | 96.06 |
| Low Cost Example | Sea Minerals | 92.58 | 94.27 | 92.76 | 93.65 |
| Control (LRI) | Bamboo Blossom | 73.27 | 95.34 | 66.34 | 95.41 |
| Low Cost Example | Bamboo Blossom | 91.93 | 95.68 | 89.58 | 95.45 |
| Control (LRI) | Champagne Toast | 95.09 | 95.81 | 90.03 | 95.67 |
| Low Cost Example | Champagne Toast | 95.81 | 95.85 | 94.12 | 95.95 |
| Control (LRI) | Warm and Cozy | 82.50 | 95.97 | 92.67 | 96.19 |
| Low Cost Example | Warm and Cozy | 96.32 | 96.46 | 96.13 | 96.32 |

As can be seen from Table 2, the clarity of each of the fragrance compositions utilizing Low Cost Microemulsion Composition #2 were generally better than or equal to the fragrance compositions utilizing the Control LRI surfactant, producing good results, and improving on freeze-thaw and heat aging properties.

### Inventive Example 3

The microemulsion compositions of the present example included each of the components and amounts as set forth in Table 8 (referred to as Bio-based Microemulsion—Composition #3).

### Example 3

### Bio-based Microemulsion Composition #3

| Component | Weight/Weight Percentage (%) |
|---|---|
| Brij ™ IC20-70 | 4.645 |
| Poly Suga ^{®}Mulse D9 | 0.75 |
| Linsurf LA-9 85 NAT | 1.175 |
| Fragrance | 2.00 |
| Preservative | 0.65 |
| 1% Antifoam Solution | 0.50 |
| DI Water | 90.28 |

The bio-based branched ethoxylated alcohol having a hydrophilic-lipophilic balance of greater than 6 (Brij IC20-70), a bio-based sorbitan oleate decyl glucoside (Poly Suga@Mulse D9), and a linear ethoxylated alcohol (Linsurf LA-9 85 NAT) were weighed into a glass beaker, and agitated to form a homogeneous solution. The fragrance was then added and mixed until homogeneous. The solution was agitated with a magnetic stir bar at 500 rpm. A preservative, an antifoam, and Deionized (DI) water were then added sequentially under agitation, to form the Bio-based Microemulsion Composition #3. The Bio-based Microemulsion Composition #3 was mixed for a minimum of 30 minutes at 500 rpm. The Bio-based Microemulsion Composition #3 was observed to have generally very good transparency and did not likely need a full 30 minutes of agitation.

As can be seen from Table 3, the clarity of each of the fragrance compositions utilizing Bio-based Microemulsion Composition #3 were generally better than or equal to the fragrance compositions utilizing the Control LRI surfactant, producing good results, and improving on freeze-thaw and heat aging properties.

TABLE 3
Bio-based Inventive Examples #11-#15

| | | | RT | 2 Mo | 2 Mo |
|---|---|---|---|---|---|
| | | F/T | (21° C.) | 45° C. | 5° C. |
| Microemulsion Composition | Fragrance | Haze L | Haze L | Haze L | Haze L |
| Control (LRI) | White Cashmere | 88.29 | 95.80 | 91.98 | 95.93 |
| Bio-based Example | White Cashmere | 96.00 | 96.07 | 96.07 | 95.98 |
| Control (LRI) | Sea Minerals | 83.28 | 95.98 | 87.33 | 96.06 |
| Bio-based Example | Sea Minerals | 96.21 | 96.12 | 96.21 | 96.19 |
| Control (LRI) | Bamboo Blossom | 73.27 | 95.34 | 66.34 | 95.41 |
| Bio-based Example | Bamboo Blossom | 94.49 | 95.91 | 96.02 | 95.80 |
| Control (LRI) | Champagne Toast | 95.09 | 95.81 | 90.03 | 95.67 |
| Bio-based Example | Champagne Toast | 95.63 | 95.99 | 95.39 | 95.89 |
| Control (LRI) | Warm and Cozy | 82.50 | 95.97 | 92.67 | 96.19 |
| Bio-based Example | Warm and Cozy | 96.15 | 96.14 | 96.42 | 96.44 |

The commercial control microemulsion composition is set forth in Table 4 (referred to as Control Microemulsion Composition).

### Example 4

### Control Example

### Control Microemulsion Composition

| Component | Weight/Weight Percentage (%) |
|---|---|
| Solubilisant LRI ™ | 5.55 |
| Fragrance | 2.00 |
| Preservative | 0.65 |
| 1% Antifoam Solution | 0.50 |
| DI Water | 91.30 |

The control microemulsion was prepared for comparison purposes. The control microemulsion composition was prepared in the same manner as the Partially Bio-based Microemulsion Composition #1 except Solubilisant LRI™ (referred to as “LRI”) was substituted in at 5.55% w/w as the surfactant component. Solubilisant LRI™ is a partially bio-based, non-ionic surfactant blend of PPG-26 Buteth-26 and PEG-40 Hydrogenated Castor Oil. The results of the control examples are seen in TABLES 1-3.

### Non-Inventive Examples #1-3

The microemulsion compositions of the present example included each of the components and amounts as set forth in Table 6 (referred to as Partially Bio-based Microemulsion Composition).

### Example 5

### Non-Inventive Example #1

### Non-Inventive Microemulsion Composition #1

| Component | Weight/Weight Percentage (%) |
|---|---|
| Eco Brij ™L23-69 | 6.945 |
| Fragrance | 2.00 |
| Preservative | .65 |
| 1% Antifoam Solution | .50 |
| DI Water | 89.905 |

A linear bio-based ethoxylated alcohol having a hydrophilic-lipophilic balance of greater than 6 (Eco Brij L23-69), was weighed into a glass beaker. The surfactant was agitated with a magnetic stir bar at 500 rpm. The fragrance was then charged, and mixed until homogeneous. Next a preservative, then an antifoam, and then Deionized (DI) water were added sequentially under agitation, to form the non-inventive Microemulsion Composition #1. Non-inventive Microemulsion Composition #1 was mixed for a minimum of 30 minutes at 500 rpm. The Bio-based Non-Inventive Microemulsion Composition #1 was observed to have generally poor transparency, and did not work as well as the inventive systems. The results are seen in TABLE 4.

TABLE 4
Non-Inventive Example #1

| | | | RT | 2 Mo | 2 Mo |
|---|---|---|---|---|---|
| | | F/T | (21° C.) | 45° C. | 5° C. |
| Microemulsion Composition | Fragrance | Haze L | Haze L | Haze L | Haze L |
| Control (LRI) | White Cashmere | 88.29 | 95.80 | 91.98 | 95.93 |
| Non-inventive Example #1 | White Cashmere | NA | 8.75 | NA | NA |
| Control (LRI) | Sea Minerals | 83.28 | 95.98 | 87.33 | 96.06 |
| Non-inventive Example #1 | Sea Minerals | NA | 8.46 | NA | NA |
| Control (LRI) | Bamboo Blossom | 73.27 | 95.34 | 66.34 | 95.41 |
| Non-inventive Example #1 | Bamboo Blossom | 80.88 | 89.96 | 94.95 | 94.83 |
| Control (LRI) | Champagne Toast | 95.09 | 95.81 | 90.03 | 95.67 |
| Non-inventive Example #1 | Champagne Toast | 95.50 | 95.58 | 95.86 | 95.80 |
| Control (LRI) | Warm and Cozy | 82.50 | 95.97 | 92.67 | 96.19 |
| Non-inventive Example #1 | Warm and Cozy | 96.45 | 96.53 | 96.45 | 96.40 |

### Example 6

### Non-Inventive Example #2

### Non-Inventive Microemulsion Composition #2

| Component | Weight/Weight Percentage (%) |
|---|---|
| Linsurf LA-9 85 NAT | 5.88 |
| Fragrance | 2.00 |
| Preservative | .65 |
| 1% Antifoam Solution | .5 |
| DI Water | 90.97 |

A bio-based linear ethoxylated alcohol having a hydrophilic-lipophilic balance of greater than 6 (Linsurf LA-9 85 NAT) was weighed into a glass beaker, and agitated with a magnetic stir bar at 500 rpm. The fragrance was added and mixed until homogeneous. Then a preservative, the antifoam, and the Deionized (DI) water, were sequentially added under agitation, to form the non-inventive Bio-based Microemulsion Composition #2. The Bio-based Microemulsion Composition #2 was mixed for a minimum of 30 minutes at 500 rpm. The Bio-based Microemulsion Composition #2 was observed to have generally very good transparency and did not likely need a full 30 minutes of agitation. Although initial clarity was very good, stability at 45 C and after 3 freeze-thaws showed some weakness with some of the fragrances tested.

TABLE 5
Non-Inventive Example #2

| | | | RT | 2 Mo | 2 Mo |
|---|---|---|---|---|---|
| | | F/T | (21° C.) | 45° C. | 5° C. |
| Microemulsion Composition | Fragrance | Haze L | Haze L | Haze L | Haze L |
| Control (LRI) | White Cashmere | 88.29 | 95.80 | 91.98 | 95.93 |
| Non-inventive Example #2 | White Cashmere | 95.80 | 96.11 | 96.59 | 95.96 |
| Control (LRI) | Sea Minerals | 83.28 | 95.98 | 87.33 | 96.06 |
| Non-inventive Example #2 | Sea Minerals | 93.32 | 95.82 | 93.33 | 95.70 |
| Control (LRI) | Bamboo Blossom | 73.27 | 95.34 | 66.34 | 95.41 |
| Non-inventive Example #2 | Bamboo Blossom | 89.99 | 95.48 | 91.80 | 95.48 |
| Control (LRI) | Champagne Toast | 95.09 | 95.81 | 90.03 | 95.67 |
| Non-inventive Example #2 | Champagne Toast | 92.49 | 95.70 | 93.14 | 95.66 |
| Control (LRI) | Warm and Cozy | 82.50 | 95.97 | 92.67 | 96.19 |
| Non-inventive Example #2 | Warm and Cozy | 96.47 | 96.40 | 96.37 | 96.29 |

### Example #7

### Non-Inventive Example #3

### Non-Inventive Microemulsion Composition #3

| Component | Weight/Weight Percentage (%) |
|---|---|
| Poly Suga Mulse D9 | 5.00 |
| Fragrance | 2.00 |
| Preservative | .65 |
| 1% Antifoam Solution | .50 |
| DI Water | 91.85 |

A bio-based sorbitan oleate decyl glucoside (Poly Suga®Mulse D9) was weighed into a glass beaker. The surfactant was agitated with a magnetic stir bar at 500 rpm. The fragrance was added, and mixed until homogeneous. Then the preservative, the antifoam, and the Deionized (DI) water were added sequentially under agitation, to form the non-inventive Bio-based Microemulsion Composition #3. The Bio-based Microemulsion Composition #3 was mixed for a minimum of 30 minutes at 500 rpm. The Bio-based Microemulsion Composition #3 was observed to have varying degrees of transparency, and did not work as well as other inventive systems. Results are seen in TABLE 6.

TABLE 6
Non-Inventive Example #3.
Non-Inventive Microemulsion Composition #3.

| | | | RT | 2 Mo | 2 Mo |
|---|---|---|---|---|---|
| | | F/T | (21° C.) | 45° C. | 5° C. |
| Microemulsion Composition | Fragrance | Haze L | Haze L | Haze L | Haze L |
| Control (LRI) | White Cashmere | 88.29 | 95.80 | 91.98 | 95.93 |
| Non-inventive Example #3 | White Cashmere | 65.20 | 61.66 | 42.44 | 59.23 |
| Control (LRI) | Sea Minerals | 83.28 | 95.98 | 87.33 | 96.06 |
| Non-inventive Example #3 | Sea Minerals | 24.80 | 23.58 | 61.92 | 51.04 |
| Control (LRI) | Bamboo Blossom | 73.27 | 95.34 | 66.34 | 95.41 |
| Non-inventive Example #3 | Bamboo Blossom | 15.18 | 16.66 | 16.50 | 15.95 |
| Control (LRI) | Champagne Toast | 95.09 | 95.81 | 90.03 | 95.67 |
| Non-inventive Example #3 | Champagne Toast | 90.53 | 91.92 | 12.58 | 91.49 |
| Control (LRI) | Warm and Cozy | 82.50 | 95.97 | 92.67 | 96.19 |
| Non-inventive Example #3 | Warm and Cozy | 96.24 | 96.15 | 96.55 | 95.73 |

### Generalized Statements of the Disclosure

The following numbered statements provide a general description of the disclosure and are not intended to limit the appended claims.
Statement 1. A surfactant or surfactant blend is provided that is particularly useful in micro-emulsions with oils/fragrances/etc. of high quality, at the same or similar use levels of Solubilisant LRI™, that may be obtained at a cost of under 10%/lb, more preferably under a cost of 30%/lb, and most preferably under a cost of 40%/lb compared to microemulsions that utilize Solubilisant LRI™.
Statement 2. A surfactant or surfactant blend according to Statement 1 is provided that includes: a branched alcohol ethoxylate at a level of about 2% or more based on the total surfactant amount, the branching being defined as having at least one methyl group pendant to the main hydrocarbon chain, and which may also include PPG (polypropylene glycol) chains for introducing multiple branches onto the hydrophobe portion of the surfactant.
Statement 3. A surfactant or surfactant blend according to Statements 1 and 2 is provided wherein the ethoxylation is comprised of standard polymerized EO (ethylene oxide) chains, and/or of bio-based EO chains.
Statement 4. A surfactant or surfactant blend according to Statements 1-3 is provided, wherein the blend optionally includes an ethoxylated HCO (hydrogenated castor oil), or castor oil (CO), or other branched hydrophobe at a level of about 0.1% or more based on the total surfactant amount, which may be comprised of standard polymerized EO chains, or of naturally derived (bio-based) EO chains, and having an average EO chain content of 40 or greater than 40 units.
Statement 5. A surfactant or surfactant blend according to Statements 1-4 is provided, wherein the blend optionally includes a bio-based sorbitan oleate decyl glucoside cross polymer with an HLB of about 6 or greater, or other bio-based alkyl glucosides, sorbitan esters, and the like, with an HLB of 6 or greater, at a concentration of .1%-98% based on total surfactant weight, and/or a bio-based low molecular weight linear surfactant with an HLB of about 6 or greater at a level of .1% to 98%, based on total surfactant weight (LA 85 NAT).
Statement 6. A surfactant or surfactant blend according to Statements 1-5 is provided, wherein the total renewable bio-based content of the component(s) is greater than about 80% w/w, and more preferably greater than about 97% w/w, and most preferably at greater than about 98% w/w or 99% w/w.
Statement 7. A surfactant or surfactant blend according to Statements 1-6 is provided, wherein at least one of the components has an average polyethylene glycol (PEG) content of 5 units or greater, more preferably about 6 units or greater, and most preferably about 7 units or greater.
Statement 8. An emulsified oil product or microemulsion of Statements 1-7 is provided wherein the surfactant level is 20% or lower, more preferably 15% or lower, and most preferably 10% or lower, based on a 100% formula composition finished product.
Statement 9. An emulsified oil product or microemulsion according to Statement 8 is provided, wherein at least one of the surfactants(s) contains a hydrophobe with branched aliphatic, and/or “comb” structured aliphatic, and/or cycloaliphatic, and/or aromatic groups, and is not an alkyl phenol ethoxylate.
Statement 10. An emulsified oil product or microemulsion according to Statements 8-9 is provided, wherein the VOC content is at about 10% or below, more preferably 6% or below, and most preferably with no added VOC, not counting the fragrance or other oil, based on a 100% formula composition.
Statement 11. An emulsified oil product or microemulsion according to Statements 8-10 is provided, wherein a mono or greater-functional alcohol is optionally present.
Statement 12. An emulsified oil product or microemulsion according to Statements 8-11 is provided, wherein the ratio of total emulsifier to oil ratio is preferably below 6:1, more preferably below 5:1, and most preferably below about 3:1.
Statement 13. An emulsified oil product or microemulsion according to Statements 8-12 is provided, wherein the L value of the initial emulsion, the initial emulsion after 3 freeze-thaw cycles, the initial emulsion when stored at room temperature (21° C.) for 3 months or more, and the initial emulsion when stored at 45° C. for 2 months or more, is preferably within about 1%, and more preferably within about .5% of the same composition using Solubilisant LRI™ in its place at a 2.5:1 surfactant to oil ratio, or other similar ratios.
Statement 14. An emulsified oil product or microemulsion according to Statements 8-13 is provided, further comprising an anionic, and/or cationic, and/or amphoteric surfactant.
Statement 15. An emulsified oil product or microemulsion according to Statements 8-14 is provided, wherein high shear methods may be used, but are not necessary to produce the emulsified oil product or microemulsion.
Statement 16. An emulsified oil product or microemulsion according to Statements 8-15 is provided, wherein the average emulsion particle size is below 80 nm, more preferably below 60 nm, and most preferably below 40 nm.
Statement 17. An emulsified oil product or microemulsion according to Statements 8-16 is provided, wherein the fragrance (perfume) or oil component is present at 30% or less, more preferably 15% or less, and most preferably at 10% or less based on final formulation weight.
Statement 18. An emulsified oil product or microemulsion according to Statements 8-17 is provided, wherein antifoam, preservative, and other components provided herein may be present at up to 20% of the total formula weight.
Statement 19. An emulsified oil product or microemulsion according to Statements 8-18 is provided, wherein the antioxidant such as citric acid prevents discoloration over aging periods at various temperatures.
Statement 20. An emulsified oil product or microemulsion according to Statements 8-19 is provided, having varying degrees of water removed on initial manufacture, so that the material can be transported at low cost, and water can be added at another site to produce a final product.
Statement 21. A microemulsion composition is provided that includes:
at least one bio-based branched ethoxylated alcohol or at least one non-bio-based branched ethoxylated alcohol;
optionally, at least one hydrogenated castor oil ethoxylate or a derivative thereof;
at least one active ingredient; and
water,
wherein the microemulsion composition exhibits a transmittance substantially equal to or greater than a microemulsion composition that includes or consists essentially of butoxypropan-2-yloxy)ethanol and PEG-40 hydrogenated castor oil.
Statement 22. A microemulsion composition according to Statement 21 is provided, wherein the at least one non-bio-based hydrogenated castor oil ethoxylate or a derivative thereof is present at a level of at least about 0.1% w/w or more based on the total weight of the microemulsion composition.
Statement 23. A microemulsion composition according to Statements 21-22 is provided, wherein at least one non-bio-based hydrogenated castor oil ethoxylate or a derivative thereof includes 40 or more repeating units of ethylene glycol.
Statement 24. A microemulsion composition according to Statements 21-23 is provided, wherein the at least one active ingredient is a fragrance selected from the group consisting of White Cashmere, Sea Minerals, Bamboo Blossom, Champagne Toast, Warm and Cozy, or other fragrance/oil.
Statement 25. A microemulsion composition according to Statements 21-24 is provided, having an average emulsion particle size of 80 nm or less.
Statement 26. A microemulsion composition according to Statements 21-25 is provided, further comprising deionized water, at least one antifoam solution, at least one preservative, ethanol, or a combination thereof.
Statement 27. A microemulsion composition is provided comprising:
at least one bio-based branched ethoxylated alcohol or at least one non-bio-based branched ethoxylated alcohol;
optionally, at least one hydrogenated castor oil ethoxylate or a derivative thereof;
at least one active ingredient; and
water,
wherein the microemulsion composition exhibits a transmittance substantially equal to or greater than a microemulsion composition that includes or consists essentially of butoxypropan-2-yloxy)ethanol and PEG-40 hydrogenated castor oil.
Statement 28. A microemulsion composition according to Statement 27 is provided, wherein the at least one bio-based ethoxylated alcohol and at least one bio-based alkyl glucoside each exhibit a hydrophilic-lipophilic balance (HLB) of at least about 6.
Statement 29. A microemulsion composition according to Statements 27-28 is provided, comprising:
a bio-based branched ethoxylated isocetyl alcohol;
optionally, a bio-based linear ethoxylated alcohol; and
optionally, a bio-based sorbitan oleate decyl glucoside.
Statement 30. A microemulsion composition according to Statements 27-29 is provided, having a ratio of bio-based branched ethoxylated isocetyl alcohol to bio-based linear ethoxylated alcohol to bio-based sorbitan oleate decyl glucoside is about 80:5:15 to 35:20:45.
Statement 31. A microemulsion composition according to Statements 27-30 is provided, further comprising deionized water, at least one antifoam solution, at least one preservative, and optionally ethanol, or a combination thereof.
Statement 32. A microemulsion composition according to Statements 27-31 is provided, wherein the at least one active ingredient is a fragrance selected from the group consisting of White Cashmere, Sea Minerals, Bamboo Blossom, Champagne Toast, Warm and Cozy, or other fragrance/oil.
Statement 33. A microemulsion composition according to Statements 27-32 is provided, having an average emulsion particle size of 80 nm or less.
Statement 34. A microemulsion composition according to Statements 27-33 is provided, comprising a total bio-based content of at least about 97% w/w based on the total weight of the microemulsion composition.
Statement 35. A microemulsion composition is provided comprising:
at least one branched ethoxylated alcohol;
optionally, at least one hydrogenated castor oil or derivative thereof including 40 or more repeating units of ethylene glycol;
at least one active ingredient; and
water,
wherein the microemulsion composition exhibits a transmittance substantially equal to or greater than a microemulsion composition that consists essentially of 2-(1-butoxypropan-2-yloxy)ethanol and PEG-40 hydrogenated castor oil.
Statement 36. A microemulsion composition according to Statement 35 is provided, wherein the castor oil or a derivative thereof is a polyethylene glycol derivative of hydrogenated castor oil including 40 or more repeating units of ethylene glycol.
Statement 37. A microemulsion composition according to Statements 35-36 is provided, wherein the at least one active ingredient is a fragrance selected from the group consisting of White Cashmere, Sea Minerals, Bamboo Blossom, Champagne Toast, Warm and Cozy, or other fragrance/oil.
Statement 38. A microemulsion composition according to Statements 35-37 is provided, having an average emulsion particle size of 80 nm or less.
Statement 39. A microemulsion composition is provided that is of high quality and exhibits lightness and transmittance at the same or similar use levels of microemulsion compositions that include Solubilisant LRI™, that may be obtained at a cost of under 10%/lb, more preferably under a cost of 30%/lb, and most preferably under a cost of 40%/lb compared to microemulsions that utilize Solubilisant LRI™.
Statement 40. A microemulsion composition according to any of the preceding statements is provided that includes: a branched alcohol ethoxylate at a level of about 2% or more based on the total surfactant amount, the branching being defined as having at least one methyl group pendant to the main hydrocarbon chain, and which may also utilize PPG (polypropylene glycol) chains for introducing multiple branches onto the hydrophobe portion of the surfactant.
Statement 41. A microemulsion composition according to any of the preceding statements is provided wherein the ethoxylation is comprised of standard polymerized EO (ethylene oxide) chains, and/or of bio-based EO chains.
Statement 42. A microemulsion composition according to any of the preceding statements is provided, wherein the blend optionally includes an ethoxylated HCO (hydrogenated castor oil) or castor oil (CO) at a level of about 2% or more based on the total surfactant amount, which may be comprised of standard polymerized EO chains, or of naturally derived (bio-based) EO chains, and having an average EO chain content of 40 or more units.
Statement 43. A microemulsion composition according to any of the preceding statements is provided that includes a bio-based sorbitan oleate decyl glucoside cross polymer with an HLB of about 6 or greater, or other bio-based alkyl glucosides, sorbitan esters, and the like, with an HLB of 6 or greater, at a concentration of .1%-98% based on total surfactant weight, and/or a bio-based linear surfactant with an HLB of about 6 or greater at a level of .1% to 98%, based on total surfactant weight (LA 85 NAT).
Statement 44. A microemulsion composition according to any of the preceding statements is provided wherein the total renewable bio-based content of the component(s) is greater than about 80% w/w, and more preferably greater than about 97% w/w, and most preferably at greater than about 98% w/w or 99% w/w.
Statement 45. A microemulsion composition according to any of the preceding statements is provided, wherein at least one of the components(s) has an average polyethylene glycol (PEG) content of 5 units or greater, more preferably about 6 units or greater, and most preferably about 7 units or greater.
Statement 46. A method of preparing a microemulsion composition is provided comprising:
forming an oil phase comprising the steps of:
   combining at least one bio-based ethoxylated alcohol surfactant, and at least one active ingredient to form a solution; and
   optionally, mixing the surfactant solution with at least one non-bio-based hydrogenated castor oil ethoxylate or a derivative thereof;
forming an aqueous phase comprising the step of mixing deionized water with one or more of a preservative, antifoam solution, or ethanol to for the aqueous phase;
blending the oil phase and aqueous phase under low shear to form the microemulsion composition.
Statement 47. A method according to statement 46 is provided, wherein the at least one active ingredient is a fragrance selected from the group consisting of White Cashmere, Sea Minerals, Bamboo Blossom, Champagne Toast, Warm and Cozy, or other fragrance/oil.

We claim:

1. A microemulsion composition comprising:
at least one bio-based branched ethoxylated alcohol or at least one non-bio-based branched ethoxylated alcohol;
optionally, at least one hydrogenated castor oil ethoxylate or a derivative thereof;
at least one active ingredient; and
water,
wherein the microemulsion composition exhibits a transmittance substantially equal to or greater than a microemulsion composition that consists essentially of 2-(1-butoxypropan-2-yloxy)ethanol and PEG-40 hydrogenated castor oil.

2. The microemulsion composition of claim 1, wherein the at least one hydrogenated castor oil ethoxylate or a derivative thereof is present at a level of at least about 0.1% w/w based on the total weight of the microemulsion composition.

3. The microemulsion composition of claim 1, wherein the at least one hydrogenated castor oil ethoxylate or a derivative thereof includes 40 or more repeating units of ethylene glycol.

4. The microemulsion composition of claim 1, wherein the at least one active ingredient is a fragrance selected from the group consisting of White Cashmere, Sea Minerals, Bamboo Blossom, Champagne Toast, Warm and Cozy, or other fragrance/oil.

5. The microemulsion composition of claim 1, having an average emulsion particle size of 80 nm or less.

6. The microemulsion composition of claim 1, further comprising optionally at least one antifoam solution, at least one preservative, ethanol, or a combination thereof.

7. A microemulsion composition comprising:
at least one bio-based branched ethoxylated alcohol;
optionally, at least one bio-based alkyl glucoside alone or in combination with at least one linear ethoxylated alcohol;
water;
at least one active ingredient,
wherein the microemulsion composition exhibits a transmittance substantially equal to or greater than a microemulsion composition that consists essentially of 2-(1-butoxypropan-2-yloxy)ethanol and PEG-40 hydrogenated castor oil.

8. The microemulsion composition of claim 7, wherein the at least one bio-based ethoxylated alcohol and at least one bio-based alkyl glucoside each exhibit a hydrophilic-lipophilic balance (HLB) of at least about 6.

9. The microemulsion composition of claim 7, comprising:
a bio-based branched ethoxylated isocetyl alcohol;
a bio-based linear ethoxylated alcohol; and
a bio-based sorbitan oleate decyl glucoside.

10. The microemulsion composition of claim 9, having a ratio of bio-based branched ethoxylated isocetyl alcohol to bio-based linear ethoxylated alcohol to bio-based sorbitan oleate decyl glucoside is about 80:5:15 to 35:20:45.

11. The microemulsion composition of claim 7, further optionally comprising at least one antifoam solution, at least one preservative, ethanol, or a combination thereof.

12. The microemulsion composition of claim 7, wherein the at least one active ingredient is a fragrance selected from the group consisting of White Cashmere, Sea Minerals, Bamboo Blossom, Champagne Toast, Warm and Cozy, or other fragrance/oil.

13. The microemulsion composition of claim 7, having an average emulsion particle size of 80 nm or less.

14. The microemulsion composition of claim 7, comprising a total bio-based content of at least about 97% w/w based on the total weight of the microemulsion composition.

15. The microemulsion composition of claim 7, wherein the at least one bio-based branched ethoxylated alcohol is present in an amount of at least about 1.0% w/w based on a total weight percent of the at least one bio-based branched ethoxylated alcohol and at least one bio-based alkyl glucoside alone or in combination at least one linear ethoxylated alcohol.

16. A microemulsion composition comprising:
at least one branched ethoxylated alcohol;
at least one hydrogenated castor oil or derivative thereof comprising at least 40 or more repeating units of ethylene glycol;
at least one active ingredient; and
water,
wherein the microemulsion composition exhibits a transmittance substantially equal to or greater than a microemulsion composition that consists essentially of 2-(1-butoxypropan-2-yloxy)ethanol and PEG-40 hydrogenated castor oil.

17. The microemulsion composition of claim 15, wherein the at least one hydrogenated castor oil or a derivative thereof is a polyethylene glycol derivative of hydrogenated castor oil.

18. The microemulsion composition of claim 15, wherein the at least one active ingredient is a fragrance selected from the group consisting of White Cashmere, Sea Minerals, Bamboo Blossom, Champagne Toast, Warm and Cozy, or other fragrance/oil.

19. The microemulsion composition of claim 15, having an average emulsion particle size of 80 nm or less.
